(19) United States
(12) Patent Application Publication
(10) Pub. No.: US 2026/0224293 A1
(43) Pub. Date: Aug. 6, 2026

(54) COMBINED CURVE TEMPERATURE AND SHAPE SENSOR

(71) Applicant: Magnisity Ltd., Hod-HaSharon (IL)

(72) Inventors: Ron BARAK, Tel-Aviv (IL); Benjamin GREENBURG, Hod-HaSharon (IL); Amit COHEN, Ramat Gan (IL)

(73) Assignee: Magnisity Ltd., Hod-HaSharon (IL)

(21) Appl. No.: 19/154,806

(22) PCT Filed: Feb. 5, 2024
(86) PCT No.: PCT/IL2024/050134

§ 371 Date: Aug. 8, 2025

Related U.S. Application Data
(60) Provisional application No. 63/483,811, filed on Feb. 8, 2023.

(51) Int. Cl. A61B 34/20; A61B 17/00; A61B 18/00; A61B 18/02; A61M 25/01

(52) U.S. Cl. CPC A61B 34/20; A61B 18/02; A61B 2017/00115; A61B 2018/00577; A61B 2018/00791; A61B 2018/0212; A61B 2034/2061; A61M 2025/0166

(57) ABSTRACT

The invention relates to system and methods for combined curve temperature and shape sensing and, more particularly, but not exclusively, to system and methods for combined curve temperature and shape sensing in an interventional elongated device. An aspect of some embodiments of the invention relates to monitoring a temperature within a body of a patient before, while and/or after providing a temperature dependent treatment. In some embodiments, the monitoring comprises receiving a plurality of temperature measurement data from a plurality of sensors located along an elongated interventional device from one or more elongated interventional devices.

### RELATED APPLICATION/S

This application claims the benefit of priority of U.S. Provisional Patent Application No. 63/483,811 filed on 8 Feb. 2023, the contents of which are incorporated herein by reference in their entirety.

### FIELD AND BACKGROUND OF THE INVENTION

The present invention, in some embodiments thereof, relates to system and methods for combined curve temperature and shape sensing and, more particularly, but not exclusively, to system and methods for combined curve temperature and shape sensing in an interventional elongated device.

Ablation and cryoablation methods may cause damage to patients due to extreme temperatures in which these treatments are provided. Also, providing the wrong temperature during such processes may cause ineffective treatment. Therefore, monitoring of the temperature may be beneficial to avoid excessive heat or cold. When ablation and cryoablation treatments are provided via an endoscope/catheter, the temperature at the treatment location and along the endoscope/catheter may be difficult to monitor. Providing extreme temperatures to a remote location via the endoscope/catheter may be harmful and/or distorting for various components of the endoscope and/or catheter.

Certain Electromagnetic (EM) tracking systems use DC magnetometers to sense low-frequency EM fields for position and orientation tracking. One application of such tracking systems is for EM position and shape sensing of a medical device, such as a fully shape tracked endoscope.

U.S. Pat. No. 11,712,309B2, titled “MAGNETIC FLEXIBLE CATHETER TRACKING SYSTEM AND METHOD USING DIGITAL MAGNETOMETERS” discloses a system for magnetic tracking of a flexible catheter device or another flexible elongated device, the system comprising: at least one generator, each configured to generate an alternating magnetic field wherein each generated magnetic field has a determined source amplitude and frequency; a device comprising: a flexible tube; a plurality of sensors, the sensors are located along the flexible tube, each configured to communicate sensed values of a local magnetic field, wherein the sensed values are at least partially due to the generated magnetic field; and a host server configured to: receive the sensed local magnetic field values from the corresponding sensors; and calculate, based on the magnetic field values and the determined source amplitude and frequency, a localization of the flexible tube, wherein the host server is optionally included in a controller of the sensors.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.
Example 1. A system for combined curve temperature and shape sensing, comprising:
a. at least one elongated interventional device, comprising:
   i. an elongated flexible portion; and
   ii. a curve sensor located along said elongated flexible portion; said curve sensor configured for sensing a shape of said elongated flexible portion and a temperature along said elongated flexible portion;
b. a processing/controlling module comprising instructions for:
   iii. receiving from said curve sensor one or more measurement values of location and temperature along said curve of said elongated flexible portion;
   iv. based on said received one or more measurement values, calculating a shape and a continuous temperature curve along said curve of said elongated flexible portion.
Example 2. The system according to example 1, further comprising displaying said calculated shape and said continuous temperature curve on a display.
Example 3. The system according to example 1 or example 2, wherein the curve sensor comprises a plurality of sensor elements configured for measuring said one or more measurement values of location and temperature along said curve of said elongated flexible portion.
Example 4. The system according to any one of examples 1-3, wherein one or more of said plurality of sensor elements are configured to communicate with said processing/controlling module via a mutual bus.
Example 5. The system according to any one of examples 1-4, wherein said processing/controlling comprises instructions for calibrating said sensor elements based on said received temperature measurement values.
Example 6. The system according to any one of examples 1-5, wherein said processing/controlling module further comprises instructions for calculating a volumetric temperature map of an organ based on one or more calculated shape-temperature curves and thermal diffusivity properties of said organ.
Example 7. The system according to any one of examples 1-6, wherein said plurality of sensor comprise one or more of temperature sensors, hall-effect sensors, magneto-resistive sensors and magneto-inductive sensors.
Example 8. The system according to any one of examples 1-7, further comprising said display.
Example 9. The system according to any one of examples 1-8, wherein said displaying comprises displaying said continuous temperature curve as a sphere.
Example 10. The system according to any one of examples 1-9, wherein said displaying comprises displaying said continuous temperature curve by coloring said shape tracked device in 3D
Example 11. A method for combined curve temperature and shape sensing, comprising:
a. receiving from said curve sensor one or more measurement values of location and temperature along said curve of said elongated flexible portion;
b. based on said received one or more measurement values, calculating a shape and a continuous temperature curve along said curve of said elongated flexible portion.
Example 12. The method according to example 11, further comprising displaying said calculated shape and said continuous temperature curve on a display.
Example 13. A system for combined curve temperature and shape sensing, comprising:
a. at least one elongated interventional device, comprising:
   i. an elongated flexible portion; and
   ii. a curve sensor located along said elongated flexible portion; said curve sensor configured for sensing a shape of said elongated flexible portion and a temperature along said elongated flexible portion;
b. a processing/controlling module comprising instructions for:
   iii. receiving from said curve sensor one or more measurement values of location and temperature along said curve of said elongated flexible portion;
   iv. based on said received one or more measurement values, calculating a shape and a temperature along said curve of said elongated flexible portion.
Example 14. The system according to example 13, further comprising displaying said calculated shape and said temperature on a display.
Example 15. The system according to example 13 or example 14, wherein the curve sensor comprises a plurality of sensor elements configured for measuring said one or more measurement values of location and temperature along said curve of said elongated flexible portion.
Example 16. The system according to any one of examples 13-15, wherein one or more of said plurality of sensor elements are configured to communicate with said processing/controlling module via a mutual bus.
Example 17. The system according to any one of examples 13-16, wherein said processing/controlling comprises instructions for calibrating said sensor elements based on said received temperature measurement values.
Example 18. The system according to any one of examples 13-17, wherein said processing/controlling module further comprises instructions for calculating a volumetric temperature map of an organ based on one or more calculated shape-temperature curves and thermal diffusivity properties of said organ.
Example 19. The system according to any one of examples 13-18, wherein said plurality of sensors comprise one or more of temperature sensors, hall-effect sensors, magneto-resistive sensors and magneto-inductive sensors.
Example 20. The system according to any one of examples 13-19, further comprising said display.
Example 21. The system according to any one of examples 13-20, wherein said displaying comprises displaying said continuous temperature curve as a sphere.
Example 22. The system according to any one of examples 13-21, wherein said displaying comprises displaying said continuous temperature curve by coloring said shape tracked device in 3D.
Example 23. A method for combined curve temperature and shape sensing, comprising:
a. receiving from said curve sensor one or more measurement values of location and temperature along said curve of said elongated flexible portion;
b. based on said received one or more measurement values, calculating a shape and a temperature along said curve of said elongated flexible portion.
Example 24. The method according to example 23, further comprising displaying said calculated shape and said temperature on a display.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer and may not be feasible or practical for performing purely manually, by a human expert. A human expert who wanted to manually perform similar tasks might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic representation of an exemplary system 100 for combined curve temperature and shape sensing, according to some embodiments of the invention;

FIG. 2 is a schematic representation of an exemplary energy sphere 202 around a tip of displayed representation 116 of the elongated flexible portion 106, according to some embodiments of the invention;

FIG. 3 is a schematic representation of two exemplary displayed representations 302a/302b of two respective tracked devices, according to some embodiments of the invention; and

FIG. 4 is a flowchart of an exemplary method according to some embodiments of the invention.

### DESCRIPTION OF SPECIFIC EMBODIMENTS OF THE INVENTION

The present invention, in some embodiments thereof, relates to system and methods for combined curve temperature and shape sensing and, more particularly, but not exclusively, to system and methods for combined curve temperature and shape sensing in an interventional elongated device.

### Overview

An aspect of some embodiments of the invention relates to monitoring a temperature within a body of a patient before, while and/or after providing a temperature dependent treatment. In some embodiments, the monitoring comprises receiving a plurality of temperature measurement data from a plurality of sensors located along an elongated interventional device from one or more elongated interventional devices. In some embodiments, two or more elongated interventional devices are used at a same time. In some embodiments, the temperature data is analyzed and utilized to generate one or more outputs. In some embodiments, exemplary outputs include one or more of: a temperature curve as sensed along the elongated interventional device, a tridimensional (3D) temperature map of one or more locations and/or organs, a four-dimensional (4D) temperature map of one or more locations and/or organs, a real-time temperature monitoring of one or more locations and/or organs while a dedicated treatment is being provided.

An aspect of some embodiments of the invention relates to a system and methods for combined curve temperature and shape sensing (which includes one or more of position and shape sensing, curve sensing, curve tracking, full shape sensing and any combination thereof- and any of the terms used below herein refers to any type of sensing mentioned here), including an interventional device having an elongated flexible portion and a curve sensor along the elongated portion. In some embodiments, the curve sensor is configured to sense at least shape and temperature along a curve of elongated portion. In some embodiments, the system comprises a processing/controlling module comprising instructions to perform one or more of: receiving from the curve sensor location and temperature measurement values along the curve of the elongated portion; based on the received values, calculating a shape and a continuous temperature curve along the curve of the elongated portion; and displaying the calculated shape, temperature and/or or any other suitable output on a display.

In some embodiments, the curve sensor comprises a plurality of sensor elements measuring location and/or temperature measurement values along the curve of the elongated portion, where one or more of the sensor elements are configured to communicate with the processing/controlling module via a mutual bus.

In some embodiments, the processing/controlling module is configured to calibrate the sensor elements based on the received temperature readings.

In some embodiments, the processing/controlling module is configured to calculate a full or partial volumetric temperature map of an organ based on calculated temperature curves and thermal diffusivity properties of the organ.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Introduction

In some embodiments, the invention relates to a system for combined curve temperature and shape sensing, including a curve temperature and shape sensor. In some embodiments, the curve sensor may include a sensor array, measuring both position and orientation and temperature at discrete sensing elements. In some embodiments, the system comprises a processing module which is configured to algorithmically compute a curve to provide a smooth shape and temperature measurement along the sensor's full curve. In some embodiments, the curve sensor may include an electromagnetic (EM) shape sensor, for example including a plurality of discrete sensors such as, for example, digital electromagnetic sensors. In some embodiments, the processing module may receive measurements of magnetic field and sensor temperature from various sensors, for example via one or more mutual digital buses, wherein multiple sensors along various locations on the curve may communicate with the processing module via a shared digital bus. In some embodiments, computed curve temperature can be displayed with varying colors, for example in real-time, along a three-dimensional representation of the curve sensor's tracked shape. In some embodiments, the computed curve temperature can find use in medical applications to support various treatments, such as ablation, cryoablation and cauterization.

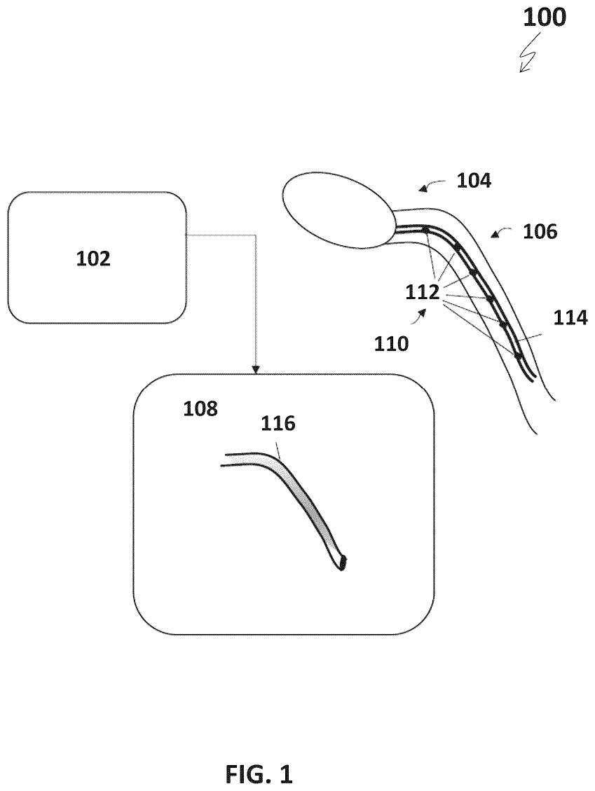

Referring now to FIG. 1, showing a schematic representation of an exemplary system 100 for combined curve temperature and shape sensing, according to some embodiments of the invention. In some embodiments, the system comprises at least one processing/controlling module 102, an interventional device 104, having an elongated flexible portion 106 and a display 108. In some embodiments, display 108 is configured for displaying a displayed representation 116 of the elongated portion 106, which can be optionally colored according to temperature (for example, in a blue-red scale). In some embodiments, the elongated flexible portion 106 includes a curve sensor module 110, configured to sense at least shape and temperature along a curve of the elongated flexible portion 106. In some embodiments, the curve sensor module 110 comprises a plurality of sensors 112. For example, the curve sensor module 110 comprises a digital EM shape sensing module, including a plurality of sensors 112. In some embodiments, the curve sensor module 110 comprises a digital sensor array, for example, an array of digital magnetometer sensors.

In some embodiments, during operation, the elongated flexible portion 106 is inserted into an anatomy of a patient. In some embodiments, the processing/controlling module 102 is configured for receiving from the curve sensor module 110 location and temperature measurement values along a curve of the elongated flexible portion 106. In some embodiments, based on the received values, the processing/controlling module 102 is configured for and/or comprises instructions for calculating one or more of shape and/or temperature variations along the elongated flexible portion 106, and displaying the calculated shape, temperature and/or or any other suitable output on the display 108.

In some embodiments, the plurality of sensors 112 comprise EM sensors configured for measuring local magnetic fields. In some embodiments, the local magnetic field readings are then used by the processing/controlling module 102 to compute locations of each one of the respective plurality of sensors 112 and/or to compute the position and shape of the full or partial elongated flexible portion 106.

In some embodiments, the plurality of sensors 112 share a digital bus 114. In some embodiments, each sensor from the plurality of sensors 112, for example a digital magnetometer sensor, comprises, for example, an integrated circuit (IC). In some embodiments, the digital bus 114 comprises I2C (Inter-Integrated Circuit) bus, I3C, SPI (Serial Peripheral Interface) or any other suitable digital bus. In some embodiments, one or more of the plurality of sensors 112 are configured for communicating their readings through the digital bus 114 to the processing/controlling module 102, which is configured for and/or comprises instructions for using the readings to provide shape tracking of the full or partial elongated flexible portion 106 in space. In some embodiments, the curve sensor module 110 comprises hall-effect sensors, magneto-resistive sensors, magneto-inductive sensors, or any other sensors which are suitable for measuring magnetic fields.

In some embodiments, as mentioned above, the curve sensor module 110 is configured for sensing temperature along the elongated flexible portion 106. In some embodiments, the plurality of sensors 112 comprise temperature sensors configured for sensing local temperature at their locations in space. In some embodiments, temperature readings are communicated to the processing/controlling module 102 through the same digital bus 114 as independent readings or alongside with other readings, such as magnetic field readings. In some embodiments, the processing/controlling module 102 is configured for and/or comprises instructions for instructing the sensor module 110 to sample, for example periodically and/or alternately, magnetic field values and local temperature. In some embodiments, all, or some of the readings of the plurality of sensors 112 are then communicated to the processing/controlling module 102 through the same digital bus 114.

In some embodiments, the processing/controlling module 102 is configured for and/or comprises instructions for calculating dynamically and/or track, for example in real time, the shape of the elongated flexible portion 106 along its curve, for example based on the magnetic readings sensed along the elongated flexible portion 106 and/or received via the digital bus 114. In some embodiments, the processing/controlling module 102 is configured for and/or comprises instructions for calculating dynamically and/or track, for example in real time, the temperature and/or temperature variations along the curve of the elongated flexible portion 106, for example based on a plurality of sampled temperatures sensed along the elongated flexible portion 106 and/or received via the digital bus 114. In some embodiments, the discrete temperature at various locations of the plurality of sensors 112 may be displayed to a user for various applications. In some embodiments, a continuous temperature variation along the elongated flexible portion 106 is calculated by the processing/controlling module 102 and/or displayed to a user for various applications.

### Exemplary Calibration

In some embodiments, the processing/controlling module 102 is configured for and/or comprises instructions for calibrating each of the plurality of sensors 112 based on the received temperature readings. In some embodiments, as some magnetic field sensors are sensitive to temperature, in an electromagnetic (EM) tracking system, EM calibration is used to convert the measured magnetic fields into six degrees of freedom (6-DOF) or five degrees of freedom (5-DOF) locations or even three degrees of freedom (3-DOF, position-only) of the sensors. In some embodiments, when an EM sensor is sensitive to temperature, the solved position and orientation of the EM sensor may be affected by changes in temperature, which may impact the EM tracking performance. In some embodiments, to overcome inaccuracy due to temperature, a temperature-dependent EM calibration is provided and/or performed for each tracked sensor. In some embodiments, for example, multiple EM calibrations are performed at different room temperatures (for example, in a range from about 10° C. to about 40° C.). In some embodiments, various calibrations per various respective temperatures are stored and/or applied by the processing/controlling module 102 on each of the respective plurality of sensors 112, for example during operation, based on temperature readings of the calibrated sensors and/or temperature readings in vicinity of the calibrated sensors.

For example, in a scenario where two calibrations were performed, a “cold” calibration at 10° C. and a “hot” calibration at 40° C., during operation, an interpolation between the two extreme calibrations can be applied, based on a current sensor's temperature reading.

In some embodiments, for example, in order to support a wider range of calibration temperatures, a temperature-dependent calibration process may include placing the curve sensor module 110 in an oven or within any other instrument which can control the temperature to a wide range (for example, from about −40° C. to about 200° C.). In some embodiments, the temperature is then modified in a certain range and the magnetic readings of the curve sensor module 110 are monitored. In some embodiments, this can be performed, for example, while transmitting known EM fields at the position of the interventional device 104. In some embodiments, the curve sensor module 110 obtains EM field readings, which might contain some error due to changes in temperature. In some embodiments, the processing/controlling module 102 is configured for and/or comprises instructions for correcting the error by finding and/or storing EM sensing calibration parameter values corresponding to the applied temperature, for example based on known differences between the EM field readings of the curve sensor module 110 and the known EM fields. In some embodiments, during operation, the processing/controlling module 102 is configured for and/or comprises instructions for correcting the EM sensor readings by using the stored temperature-dependent EM sensing calibration parameter values, which was obtained in the temperature-dependent calibration process.

In some embodiments, a temperature-dependent calibration model can be fitted in the calibration process to model the changes with temperature in magnetic field readings. In some embodiments, the error can be modeled as a linear, quadratic, cubic, or a general polynomial function of temperature, or using any other kind of suitable temperature to magnetic field error dependency. In some embodiments, after fitting the temperature-dependent calibration model (for example, using a plurality of temperature to magnetic field readings, as described above), the model can be used during operation to correct the magnetic readings based on the real-time sensed temperatures.

In some embodiments, some temperature sensors provide temperature readings which are indicative of the true sensor temperature but may be un-calibrated to some extent. For example, a temperature sensor may report temperature with a constant error bias of 4° C. In some embodiments, a temperature sensor may not be linear and may require calibration over a range of operating temperatures. In some embodiments, the temperature sensing is calibrated in a similar manner to any of the calibration processes mentioned above. In some embodiments, in a temperature-controlled EM calibration process, the temperature of the interventional device 104 is known. However, a sensor may report an un-calibrated temperature reading, for example due to tolerances in its manufacturing process. In some embodiments, the temperature readings of a sensor may be calibrated using the known applied temperature or by externally measuring the true temperature of the sensor or of the elongated device at the position of the sensor, for example, using a thermocouple, thermal imaging, pyrometer, or any other suitable method. In some embodiments, a known temperature θᵢ is applied to one or more sensors (or to the entire sensors), but the sensor temperature reading is \tilde{θ}ᵢ which may be different (uncalibrated). In this case, a plurality of temperature pairs (θᵢ,\tilde{θ}ᵢ) are collected in the calibration process. In some embodiments, a model can then be fitted to predict the true (calibrated) temperature θᵢ from the uncalibrated temperature reading \tilde{θ}ᵢ. In some embodiments, this model can be linear, quadratic, cubic, polynomial of arbitrary degree or any other suitable model. In some embodiments, instead of explicitly applying a temperature θᵢ to one or more sensors, the sensor's temperature can be measured externally, as mentioned above. In some embodiments, there may be a difference between the temperature measured by the sensor and the temperature of the elongated device at the sensor position, for example, due to the sensor's self-heating during operation. Using one of the temperature calibration methods mentioned above, the discrepancy between the temperature measured by the sensor and the elongated device's temperature at the position of the sensor can be solved (through calibration), in cases where the elongated device's temperature is of interest. In some embodiments, this can be done by letting \tilde{θ}ᵢ be the temperature measured by the sensor and θᵢ be the temperature of interest (the temperature of the elongated device at the position of the sensor) which can be measured externally or applied explicitly as mentioned above. A calibration model can then convert between \tilde{θ}ᵢ and θᵢ as mentioned above.

### Exemplary Operation of Sensors

In some embodiments, the processing/controlling module 102 is configured for and/or comprises instructions for requesting measurements from the plurality of sensors 112 and/or for reading measurements from the plurality of sensors 112 periodically, for example in a sequential manner, via the shared digital bus 114. In some embodiments, the communication between the processing/controlling module 102 and the plurality of sensors 112 is performed in periodic cycles, where each cycle includes a series of requests from the plurality of sensors 112 followed by a series of readings of measurements requested in a previous cycle from the plurality of sensors 112, or vice versa (i.e. a series of readings followed by a series of requests). In some embodiments, a duration of such cycle may be shorter than a full sampling period of the processing/controlling module 102, for example, shorter than 1 millisecond, or shorter than 1.5 milliseconds, or shorter than 2 milliseconds. In some embodiments, the processing/controlling module 102 is configured for and/or comprises instructions for sampling the entire sensor array of the curve sensor module 110 at a rate of 1 kHz, or between 500 Hz to 5 kHz.

### Exemplary Display

In some embodiments, the processing/controlling module 102 is configured for and/or comprises instructions for displaying on the display 108 the temperatures along a displayed curve of the elongated flexible portion 106. In some embodiments, the temperatures are displayed quantitatively (for example, by text), and/or by using colors for representing various temperature values. In some embodiments, various locations along the displayed curve, for example locations corresponding to locations of each respective sensor from the plurality of sensors 112 along the elongated flexible portion 106, are marked by a color indicative of the temperature measured by the respective sensor or measured in the vicinity of the respective sensors. In some embodiments, the temperature-representing colors may vary along a scale of blue (for example, indicating cold temperature, for example, 0° C.) to red (for example, indicating hot temperature, for example, 100° C.), or along a scale of blue to green to red or using any other suitable color map, indicative of the temperatures measured along the elongated flexible portion 106. In some embodiments, the range of temperature-colors displayed may vary based on the application. For example, the colors may be adjusted to cover temperatures between −40° C. to 30° C. for cryoablation or may cover temperatures between 30° C. to 120° C. for ablation.

In some embodiments, instead of displaying a set of discrete temperatures at sensor locations, a full curve temperature of the elongated flexible portion 106 is computed and displayed to the user. For example, a smooth and/or continuous temperature variation can be fitted between the discrete sensor-measured temperatures to interpolate the temperature in-between sensor locations. In some embodiments, this can be done using a linear interpolation between sensor location, quadratic interpolation, cubic interpolation, cubic spline, PCHIP (Piecewise Cubic Hermite Interpolating Polynomial), polynomial of a certain degree or any other suitable curve fitting or interpolation method. In some embodiments, the temperature variation curve is fitted taking into account the dimensions and/or locations of the plurality of sensors 112, at which the discrete temperatures are measured along the elongated device.

In some embodiments, as mentioned above, a displayed representation 116 of the elongated flexible portion 106 can be colored according to temperature (for example, in a blue-red scale). In some embodiments, the displayed representation of the elongated flexible portion 106 is displayed by a three-dimensional representation, for example while the shape of the elongated flexible portion 106 is tracked in real-time, for example along with a color variation along the displayed representation, the color variation representing the varying temperature along the elongated flexible portion 106.

In some embodiments, the processing/controlling module 102 is configured for and/or comprises instructions for providing visual and/or vocal indications about start and/or end of ablation, for example by display 108, by LEDs, by a sound alarm, and/or by any other suitable indicator. In some embodiments, the processing/controlling module 102 is configured for and/or comprises instructions for computing and/or generating an energy dissipation sphere (for ablation or cryoablation), for example based on the curve temperature tracking of one or more temperature tracked devices. In some embodiments, the energy sphere may be displayed by display 108, thus, for example, helping a physician to monitor and control the ablation process.

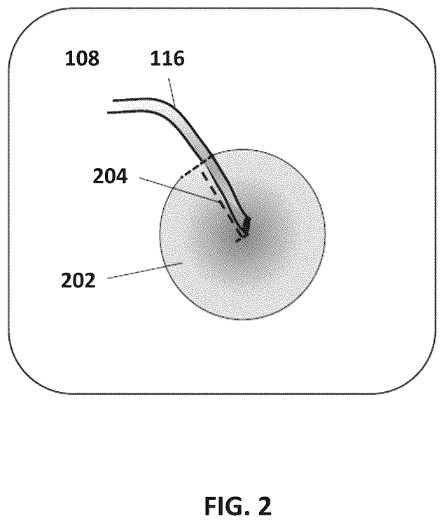

Referring now to FIG. 2, showing a schematic representation of an exemplary energy sphere 202 around a tip of displayed representation 116 of the elongated flexible portion 106, according to some embodiments of the invention. In some embodiments, the sphere 202 is optionally provided with a color or a spectrum of colors indicative of the sensed temperature. In some embodiments, the size of sphere 202 may be computed, for example, from the sensed curve temperature along the elongated flexible portion 106. In some embodiments, the processing/controlling module 102 is configured for and/or comprises instructions for determining a radius of the sphere 202, by recognizing a length 204 along representation 116, for example including the tip of the representation 116 as the center of the sphere 202, in which the temperature is significantly different, for example significantly higher, than in the rest of the representation 116. In some embodiments, the recognized length is determined as the radius of the sphere 202. In some of the calculations and representations mentioned above, the sphere is centered at the tip of the device, since the tip of the device is assumed to be the source of energy, such as an ablation catheter, or cryoablation catheter, or cautery. In this case, the tip of the device is a source of temperature (for example, high or low temperature) which may spread as a sphere which is centered at the device's tip. It can therefore be assumed that the high/low temperature spreads as a symmetrical sphere around the energy source which is centered at the device's tip (where the ablation, cryoablation, cautery or other energy source instrument may be located).

### Exemplary Calculations

In some embodiments, in an exemplary process to calculate a continuous temperature curve, the processing/controlling module 102 is configured for and/or comprises instructions for fitting a temperature curve along the elongated flexible portion 106 via the various temperature measurements at the locations of the plurality of sensors 112, so that the curve is continuous and smooth (has continuous derivatives).

In some embodiments, then, the processing/controlling module 102 is configured for and/or comprises instructions for minimizing the curvature of the temperature curve along the elongated flexible portion 106, for example, by minimizing the total of the second derivative values of the temperature curve, along the elongated flexible portion 106.

In some embodiments, the processing/controlling module 102 is configured for and/or comprises instructions for dividing the temperature curve to small segments, for example 1 mm segments, assigning to each segment a corresponding discrete temperature value, and estimating the total curvature, for example, by summing differential elements between neighboring temperature values. In some embodiments, the total curvature is minimized by any suitable optimization methods such as, for example, gradient descent or Levenberg-Marquardt. In some embodiments, optimal temperature values at a corresponding series of locations along the elongated flexible portion 106 are determined to minimize the total curvature of the temperature curve by averaging neighboring temperature values in an iterative process, optionally until optimal values are obtained.

In some embodiments, the temperature curve is estimated according to cubic splines, PCHIP (Piecewise Cubic Hermite Interpolating Polynomial), a polynomial or using any other suitable analytic or piecewise analytic function. In some embodiments, the processing/controlling module 102 is configured for and/or comprises instructions for optimizing the estimation of the temperature curve by fitting the curve via the measured temperature values at the corresponding locations and optionally minimizing the total of the second derivative values of the temperature curve, along the elongated flexible portion 106.

### Exemplary Uses of the Exemplary System 100 for Combined Curve Temperature and Shape Sensing

In some embodiments, the exemplary system 100 for combined curve temperature and shape sensing can be potentially useful in certain medical treatments, as will be further shown in the examples below. In some embodiments, the exemplary system 100 for combined curve temperature and shape sensing is used to track the temperature along a treatment device (such as an ablation or cryoablation catheter or cautery) or along a treatment carrying device (such as an endoscope with a working channel, through which certain treatment instruments can be introduced), or at the local 3D volume at the vicinity of an endoscopic target (such as a suspected lesion). In some embodiments, the exemplary system 100 for combined curve temperature and shape sensing is used in a combined temperature and shape tracking, where the shape tracking capabilities of the device are used to guide the device to a specific location inside an organ of a patient. In some embodiments, the exemplary system 100 for combined curve temperature and shape sensing is used in navigational bronchoscopy, for example using shape tracked endoscopes to guide the endoscope to a specific location inside the lungs. In some embodiments, the full temperature tracking of the exemplary system 100 for combined curve temperature and shape sensing is used to assist in delivering localized treatment through a tracked endoscope. In some embodiments, the exemplary system 100 for combined curve temperature and shape sensing is used in the case of localized ablation to track the heat dissipation of the ablation catheter as it ablates along the length of the endoscope, from the distal end back to the proximal end. In some embodiments, the temperature information generated by the exemplary system 100 for combined curve temperature and shape sensing is used in understanding the distribution of ablation energy during treatment in order to monitor and control the ablation process. In some embodiments, similarly, the exemplary system 100 for combined curve temperature and shape sensing is used in the case of localized cryoablation treatment, where tracking the temperature along the endoscope is used for understanding the distribution of cryogenic energy during treatment. In some embodiments, a potential advantage of utilizing the exemplary system 100 for combined curve temperature and shape sensing is that it can potentially aid in shortening treatment time, preventing healthy tissue from being unnecessarily ablated, localizing the treatment only at a specific point of interest (e.g., a lesion), timing energy delivery, verifying that treatment was efficiently performed (by sensing sufficient heat for sufficient time) and generally, for monitoring and controlling any localized energy treatment, according to some embodiments of the present disclosure. In some embodiments, the energy of the treatment device (such as an ablation catheter, cryoablation or cautery) is assumed to spread in a spherical symmetrical manner. In this case, by measuring the energy spread (the temperature distribution) along the curve of exemplary system 100 for combined curve temperature and shape sensing the full sphere may be reconstructed, by assuming that it is centered at the device's tip and spreads symmetrically around the device's tip, with temperature distribution as measured along the device's curve. In another embodiment the energy may be assumed to spread non-symmetrically, for example, according to a model which is derived from tissue density as measured in a registered CT scan, as mentioned in more detail below.

In some embodiments, the energy and/or temperature distribution model is used to predict the temperature at locations other than the shape tracked device, where the curve temperature is measured along the shape of the device. For example, in a spherical symmetrical energy distribution model, an energy source can be assumed to be located at the tracked tip of the device, for example, in the case of an ablation or cryoablation instrument, which may be introduced through the working channel of the device and may be assumed to extend a few millimeters towards from the location of the tip of the device. In some embodiments, such energy source may then be assumed to generate a spherical symmetrical spread of energy during operation. In some embodiments, under the assumption of such energy distribution model, by measuring the temperature along the curve of the device, a sphere of temperature is reconstructed, centered at the energy source location (near the tracked tip of the device), such that the entire energy sphere may be reconstructed using the 1D measured temperature curve along the device. In some embodiments, a potential advantage of this simplified energy distribution model is that it can be potentially useful in the case of ablation and cryoablation where the energy is roughly estimated to distribute as a symmetrical sphere, and where the 1D measured temperature along the curve of the device is sufficient to estimate the spread of that sphere.

In some embodiments, the energy source may not necessarily be assumed to be located at the tracked tip of the device or at a fixed location or at a distance relative to the tip of the device. In some embodiments, for example, in some cases an ablation or cryoablation or other treatment instrument may be introduced into the working channel of the tracked device and may extend from the tracked tip of the device further out with a variable or unknown distance. In some embodiments, the tracked device may contain an embedded imaging sensor (such as in the case of a tracked endoscope device). In this case, the camera of the device can be used to detect the treatment instrument in the image and estimate its distance from the tip of the device by analyzing the image (for example, by detecting the treatment instrument tip position and size in the image, under the assumption that the imaging sensor of the tracked device is located at a known position relative to the tracked tip of the device). In some embodiments, the location of the tip of the treatment instrument is detected using EM methods. In some embodiments, for example, the plurality of EM sensors along the tracked device is used both for tracking the shape and position of the device in space as well as for tracking the position of a tool (instrument) which is inserted into the working channel of the device, by detecting the EM distortion caused by the introduced instrument, and especially by the tip of the instrument.

In some embodiments, using a plurality of shape and temperature tracked devices, the reconstructed 3D/4D temperature map may be enhanced. For example, in the case of a spherical symmetrical energy distribution model, by combining curve temperature measurements from multiple devices, the reconstructed energy sphere may be computed with greater accuracy, since each temperature curve provides additional measurements for the energy/temperature model, and the energy distribution model (for example, a spherical symmetrical model) can combine all available measurements to find the best fit (for example, in least-squares sense) of the model onto the measurements. In some embodiments, this potentially reduces the risk of overfitting the model. In some embodiments, in order to reconstruct a simplified spherically-symmetrical energy distribution 3D temperature map, one or more shape tracked temperature curves may be used. Denote by rₖ(σ) the k-th location of the device in 3D EM transmitter coordinates, and assume that σ is a length parameter starting at the tip of the device, such that rₖ(0) is the location of the tip of the device in 3D and rₖ(L) is the proximal location of the device, where L may be the tracked length of the device in millimeters. Assuming that an ablation catheter serves as an energy source and is placed at the first location of the tip of the device: r_{src}=r₁(0). Denote by θₖ(σ) the temperature of the device at length parameter σ, which is attributed to temperature measurement at 3D position rₖ(σ). Under the assumption of a spherically-symmetrical energy distribution, it may be assumed that θ(r) is uniform for any r at a fixed distance D from the energy source r_{src}, that is: ∥r−r_{src}∥=D. The temperature may then be thought of as a function of distance from the source: θ(D). Then, the temperature measurements θₖ(σ) actually provide temperature measurements at a plurality of distances from the energy source r_{src}, since: θₖ(σ)=θ(rₖ(σ))=θ(Dₖ(σ))=θ(∥rₖ(σ)−r_{src}∥). Finally, the temperature at distance D from the source may be estimated for example as the mean estimation from all K available curve measurements:

$$Θ ( D ) = \frac{1}{K} {\sum }_{k = 1}^{K} {Θ}_{k} ( {σ}_{k} )$$

Where each σₖ is such that |rₖ(σₖ)−r_{src}∥=D.

In some embodiments, r_{src} is not necessarily assumed to be located at a certain location of the tracked tip of the device. For example, it may be assumed to be located a few millimeters ahead of a certain location of the tracked tip of the device (for example, along the tracked direction of the tip of the device).

In some embodiments, a more general energy distribution model may be assumed. For example, a parametrized model may be assumed with N-dimensional parameter vector x, such that θ(x,r) denotes the temperature at 3D location r in space, under the assumption of model parameters x. By providing curve temperatures as denoted above: θₖ(σ), the model parameters x can be searched such that the model satisfies the measurements:

$$Θ ( x , {r}_{k} ( σ ) ) = {Θ}_{k} ( σ )$$

Searching for x which best satisfies the measurements can be done using linear or non-linear local or global optimization methods, such as Gradient descent, Levenberg-Marquardt, or any other optimization method. By finding the model parameters x, the energy (temperature) can then be predicted for any r (not just for locations along the shape tracked devices), by just computing θ(x,r).

In some embodiments, the energy distribution model can be 4D, by adding the time t to the energy distribution model θ(x,r,t) and modifying the measurements equation above:

$$Θ ( x , {r}_{k}^{t} ( σ ) , t ) = {Θ}_{k}^{t} ( σ )$$

### Where

$${r}_{k}^{t} ( σ )$$

denotes the shape-tracked k-th location of the device along length parameter σ at time t and

$${Θ}_{k}^{t} ( σ )$$

denotes the temperature measurement of the k-th device along length parameter σ at time t.

In some embodiments, the general energy distribution model may take into consideration tissue information, as may be segmented from a preoperative CT scan, as mentioned in more detail below.

In any such case, by using combined position and shape tracking and temperature curve sensing, temperature curves can be used to reconstruct a local 3D/4D temperature map according to some energy distribution model, which is specifically useful for measuring the energy distribution of treatment instruments such as ablation, cryoablation or any other treatment instruments.

In some embodiments, the exemplary system 100 for combined curve temperature and shape sensing is used for temperature tracking to detect the start and stop events of a localized ablation process. For example, the processing/controlling module 102 is configured for and/or comprises instructions for detecting a rapid increase or decrease in the temperature at the tip of the endoscope, which can be attributed to the start or the end, respectively, of an ablation process.

### Exemplary Use of Multiple Devices

In some embodiments, multiple tracked devices are introduced simultaneously to an organ of a patient. For example, two or more combined shape and temperature tracked endoscopes, or catheters, or guidewires are introduced to the bronchi of a patient or other type of lumen. In some embodiments, each device is configured to be shape-temperature tracked. In some embodiments, shape tracking is used to guide each device to a specific location inside the organ of the patient, for example, inside the lungs. In some embodiments, full curve temperature tracking is used to track the temperature along the specific known curve position inside the anatomy of the patient. In some embodiments, this provides tracking of the temperature of the patient along a specific known curve inside the anatomy of the patient. In some embodiments, the 3D locations of the one or more tracked curves are known due to shape and/or position tracking and supporting navigational algorithms (for example, anatomy registration and deformation algorithms) such that the 3D locations are known in the anatomy. In some embodiments, additionally, in the setting of EM shape tracking, each of the simultaneously tracked devices is localized in the same coordinate system of a shared transmitter, such that the simultaneously tracked curves are accurate in position and orientation one relative to another and all relative to a same transmitter. In some embodiments, temperature tracking provides real-time tracking of the temperature of the organ along anatomical curves at known locations. In some embodiments, by combining information from multiple tracked temperatures at known curves in the anatomy, a temperature map of the anatomy or of the partial anatomy is reconstructed.

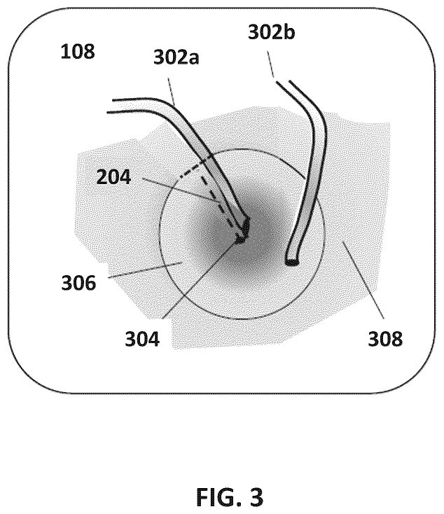

Referring now to FIG. 3, showing a schematic representation of two exemplary displayed representations 302a/302b of two respective tracked devices, according to some embodiments of the invention. In FIG. 3, a tip of displayed representation 302a is shown at a target 304. In some embodiments, a computed energy sphere 306 is displayed around the tip of representation 302a and target 304. In some embodiments, a local reconstructed three or four-dimensional (3D/4D) temperature map 308 is displayed around target 304. In some embodiments, map 308 is computed and generated by the processing/controlling module 102 as described in more detail herein. In some embodiments, sphere 306 is computed according to map 308, for example by fitting a sphere to the range in map 308 in which the temperature is significantly different, for example significantly higher, than in the rest of map 308. In other embodiments, the temperature map is displayed to the user in 3D for example using raytracing methods through the 3D temperature map, where each position in the 3D temperature map is assigned a color through a color map which is indicative to the temperature at that position.

In some embodiments, a mathematical and/or physical model of the temperature of the organ in three dimensions is utilized, as will be further explained herein.

In navigational procedures it is common that a preoperative CT scan of the anatomy of the patient is available. For example, in the case of navigational bronchoscopy, a preoperative CT scan of the patient is used during the navigational procedure to guide the physician through the lungs of the patient based on the preoperative CT scan (which is used as a map).

In some embodiments, the processing/controlling module 102 is configured for and/or comprises instructions for using the CT scan to construct a map of the anatomy, having a corresponding three-dimensional coordinate system. In some embodiments, the processing/controlling module 102 is configured for and/or comprises instructions for localizing, in the three-dimensional coordinate system of the CT scan, various tracked temperature-curves inside the anatomy of the patient. In some embodiments, various temperature-curves from various tracked devices are provided to the processing/controlling module 102. In some embodiments, the processing/controlling module 102 is configured for and/or comprises instructions for constructing a three-dimensional (or four-dimensional, that is, time dependent) temperature map of the organ by treating the various temperature-curves as boundary conditions (which can also be thought of as constraints, or partial measurements) for a three-dimensional temperature map model function. For example, in a heat steady state, the three-dimensional temperature function may be assumed to be harmonic inside the domain (that is, its Laplacian can be assumed to be zero), or harmonic in subdomains. In some embodiments, the temperature in certain location may be determined by finding, in the certain location, a temperature that match the boundary temperature curves while minimizing the model function inside the domain of the constructed three-dimensional map.

In some embodiments, the three-dimensional temperature map model function is generated, for example, using a three-dimensional polynomial function, Thin-plate splines (TPS), a dense grid representation or using any other suitable method.

In some embodiments, the system is configured for and/or comprises instructions for determining the temperature in a three-dimensional volume inside the patient's organ, which can be potentially advantageous, for example, for monitoring and controlling localized energy delivery treatments or conditions of the patient by reconstructing the temperature map from the curve measurements.

In some embodiments, the processing/controlling module 102 is configured for and/or comprises instructions for incorporating into the temperature mapping model the changes to the temperature with time, across the organ volume, which potentially enables to model the temperature map more accurately.

In some embodiments, the model includes the thermal diffusivity of different tissues inside the organ of the patient. For example, when ablating a lesion inside the lungs, different lung tissues can be modeled with different diffusivity values, for example, computed from the CT scan of the patient. In some embodiments, different organ tissues are segmented and classified, for example by image processing methods such as AI segmentation neural networks. For example, the processing/controlling module 102 is configured for and/or comprises instructions for detecting and/or classifying airways, blood-vessels, lesions, bones, pleura, diaphragm, heart, among other type of tissues. In some embodiments, this can potentially help in assigning a thermal diffusivity coefficient with any type of tissue inside an organ such as the lungs.

In some embodiments, the temperature map along space and time is modeled and updated, for example according to measurements received from the plurality of sensors 112. In some embodiments, multiple shape-temperature tracked devices are inserted in the proximity of a target lesion to be treated. For example, an endoscope may be inserted to the center of the target and a second catheter may be inserted to a chosen location (by a system or physician) near the target. In some embodiments, a navigation system aids in the guidance and navigation of the devices to the specific chosen locations, for example, relying on the tracked shapes of the devices. In some embodiments, once the devices are in place, an ablation tool is inserted through the working channel of the endoscope to the center of the target. In some embodiments, both the devices are shape-temperature tracked. In some embodiments, when the ablation tool starts to ablate the tissue, the system monitors the curve temperatures of the two (or more) tracked devices, which includes curve temperature of the primary device, the endoscope, with its distal end at the target and the secondary device (or more) at the proximity of the target. In some embodiments, the system is configured for and/or comprises instructions for reconstructing a local three or four-dimensional (3D/4D) temperature map at the proximity of the ablated target, as discussed above. In some embodiments, the tracked temperature curves at the proximity of the target allows the system to reconstruct an accurate 3D/4D temperature map at the region of the ablated target, which can be updated in time according to the temperature map model, as mentioned above. In some embodiments, the reconstructed 3D/4D temperature map is used to monitor and control the localized energy treatment process to a high degree. For example, the reconstructed 3D/4D temperature map can be used to monitor the spread of heat from the source (at the center of the lesion, or where the ablation, or cryoablation or other treatment instrument is assumed to be, or where its position is determined by tracking) in order to prevent ablation of healthy tissue at the proximity of the target or to maximize the effectiveness and efficiency of treatment for the unhealthy tissue. In some embodiments, additionally, the reconstructed 3D/4D temperature map is used to prevent damage to other important anatomical features at the proximity of the target, such as blood vessels, heart, pleura, diaphragm, or any other anatomical features which need to be protected.

In some embodiments, a potential advantage of the reconstructed 3D/4D temperature map is that it can aid in monitoring and controlling of the localized treatment.

In some embodiments, by combining the knowledge of precise location inside the anatomy with real-time tracked temperature, the system is able to deliver precise and effective localized treatment with minimal collateral damage.

### Exemplary Methods

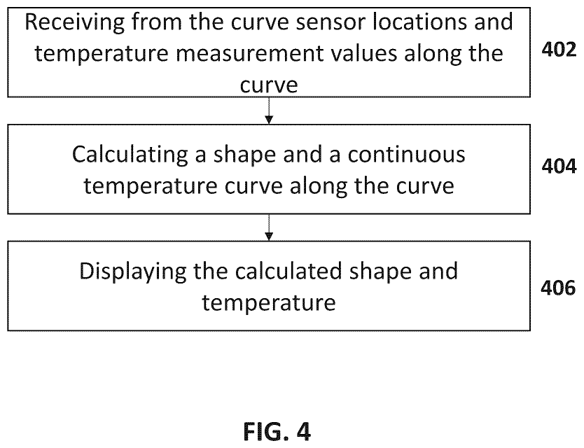

Referring now to FIG. 4, showing a flowchart of an exemplary method according to some embodiments of the invention. In some embodiments, an exemplary method comprises one or more of the following actions:
1. Receiving from the curve sensor locations and temperature measurement values along the curve (402);
2. Calculating a shape and a continuous temperature curve along the curve (404); and
3. Displaying the calculated shape and temperature (406).

The abovementioned actions are all explained and disclosed in the paragraphs above.

While throughout the disclosure digital sensors are used it should be appreciated that some of the described methods apply to any elongated device capable of measuring a plurality of temperatures along its length, for example, by using resistive temperature sensors (which may not necessarily be digital). These sensors can be combined, for example, with a fiber optics shape sensor, which is not necessarily digital. Another alternative configuration may be a curve-resistive or curve-inductive sensor, measuring curve-temperature along its length, or measuring both temperature and shape using magneto-resistive, magneto-inductive, temperature-resistive or temperature inductive properties of the sensor.

It should also be appreciated that a local 3D/4D temperature map, as described above, can be reconstructed from shape-temperature measurements which are obtained in any suitable method, not necessarily using a digital EM shape-temperature sensor, for example, using a fiber optics shape sensor in combination with temperature sensors of any kind.

While magnetic and temperature sensors are used throughout the disclosure, it should be appreciated that a digital EM shape sensor may include magnetic field sensor, IMU sensor (accelerometer and/or gyro), temperature sensor, pressure/strain sensors, or any other kind of suitable digital sensor which can be placed on a digital bus. Information from all sensors may participate in the reconstruction of a 3D/4D local temperature map to control and monitor localized treatment.

As used herein with reference to quantity or value, the term “about” means “within +10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method, or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein, and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section 10 headings are used, they should not be construed as necessarily limiting. In addition, any priority document(s) of this application is/are hereby incorporated herein by reference in its/their entirety.

What is claimed is:

1. A system for combined curve temperature and shape sensing, comprising:
a. at least one elongated interventional device, comprising:
i. an elongated flexible portion; and
ii. a curve sensor located along said elongated flexible portion; said curve sensor configured for sensing a shape of said elongated flexible portion and a temperature along said elongated flexible portion;
b. a processing/controlling module comprising instructions for:
iii. receiving from said curve sensor one or more measurement values of location and temperature along said curve of said elongated flexible portion;
iv. based on said received one or more measurement values, calculating a shape and a temperature along said curve of said elongated flexible portion.

2. The system according to claim 1, further comprising displaying said calculated shape and said temperature on a display.

3. The system according to claim 1, wherein the curve sensor comprises a plurality of sensor elements configured for measuring said one or more measurement values of location and temperature along said curve of said elongated flexible portion.

4. The system according to claim 3, wherein one or more of said plurality of sensor elements are configured to communicate with said processing/controlling module via a mutual bus.

5. The system according to claim 1, wherein said processing/controlling comprises instructions for calibrating said sensor elements based on said received temperature measurement values.

6. The system according to claim 1, wherein said processing/controlling module further comprises instructions for calculating a volumetric temperature map of an organ based on one or more calculated shape-temperature curves and thermal diffusivity properties of said organ.

7. The system according to claim 3, wherein said plurality of sensors comprise one or more of temperature sensors, hall-effect sensors, magneto-resistive sensors and magneto-inductive sensors.

8. The system according to claim 1, further comprising said display.

9. The system according to claim 2, wherein said displaying comprises displaying said continuous temperature curve as a sphere.

10. The system according to claim 2, wherein said displaying comprises displaying said continuous temperature curve by coloring said shape tracked device in 3D.

11. A method for combined curve temperature and shape sensing, comprising:
a. receiving from said curve sensor one or more measurement values of location and temperature along said curve of said elongated flexible portion;
b. based on said received one or more measurement values, calculating a shape and a temperature along said curve of said elongated flexible portion.

12. The method according to claim 11, further comprising displaying said calculated shape and said temperature on a display.
